(19) United States
(12) Patent Application Publication
(10) Pub. No.: US 2017/0245865 A1
(43) Pub. Date: Aug. 31, 2017

(54) Intralumenal Occlusion Devices Having Improved Properties

(71) Applicants: Donald K. Jones, Dripping Springs, TX (US); Vladimir Mitelberg, Austin, TX (US)

(72) Inventors: Donald K. Jones, Dripping Springs, TX (US); Vladimir Mitelberg, Austin, TX (US)

(21) Appl. No.: 15/510,908

(22) PCT Filed: Sep. 14, 2015
(86) PCT No.: PCT/US15/50048

§ 371 Date: Mar. 13, 2017

Related U.S. Application Data
(60) Provisional application No. 62/050,730, filed on Sep. 15, 2014.

(51) Int. Cl. A61B 17/12

(52) U.S. Cl. CPC A61B 17/12113; A61B 17/12145; A61B 17/1215; A61B 17/12154; A61B 2017/00526

(57) ABSTRACT

Devices, systems and methods are provided for performing embolization procedures in a desired area of the body. Systems include embodiments of embolization devices having increased durability, flexibility, conformability and surface area that include elongate primary coils that are formed from helically wound elongate initial coils which are formed from helically wound metallic wire and delivery systems used to position the embolization devices at a target location within a lumen of a mammal.

### BACKGROUND OF THE INVENTION

The field of intralumenal therapy for the treatment of vascular disease states has for many years focused on the use of many different types of therapeutic devices. While it is currently unforeseeable that one particular device will be suitable to treat all types of vascular disease states it may however be possible to reduce the number of devices used for some disease states while at the same time improve patient outcomes at a reduced cost. To identify potential opportunities to improve the efficiency and efficacy of the devices and procedures it is important for one to understand the state of the art relative to some of the more common disease states.

For instance, one aspect of cerebrovascular disease in which the wall of a blood vessel becomes weakened. Under cerebral flow conditions the weakened vessel wall forms a bulge or aneurysm which can lead to symptomatic neurological deficits or ultimately a hemorrhagic stroke when ruptured. Once diagnosed a small number of these aneurysms are treatable from an endovascular approach using various embolization devices. These embolization devices include detachable balloons, coils, polymerizing liquids, gels, foams, stents and combinations thereof.

Detachable balloons were some of the earliest embolization devices used to treat aneurysms. Under fluoroscopic guidance these balloons were positioned within the aneurysm, inflated using a radio-opaque fluid and subsequently detached from their delivery mechanism. There were numerous drawbacks encountered while using these devices such as difficulty in guiding the devices to the treatment site due to size and shape, difficulties in placing the devices within the aneurysm due to the geometry of the balloons relative to the aneurysm geometry, excessive forces generated during detachment the balloons from the delivery system, dislodging of previously place balloons and delayed deflation of the detached balloons. Examples of various detachable balloon systems attempting to address some of the aforementioned drawbacks are disclosed in U.S. Pat. No. 3,834,394 to Hunter entitled, “Occlusion Device and Method and Apparatus for Inserting the Same”, U.S. Pat. No. 4,085,757 to Pevsner entitled, “Miniature Balloon Catheter Method and Apparatus, U.S. Pat. No. 4,327,734 to White Jr. entitled, “Therapeutic Method of Use for Miniature Detachable Balloon” U.S. Pat. No. 4,364,392 to Strother entitled “Detachable Balloon Catheter”, U.S. Pat. No. 4,402,319 to Handa, entitled, “Releasable Balloon Catheter”, U.S. Pat. No. 4,517,979 to Pecenka, entitled, “Detachable Balloon Catheter”, U.S. Pat. No. 4,545,367 to Tucci entitled, “Detachable Balloon Catheter and Method of Use”, U.S. Pat. No. 5,041,090 to Scheglov entitled, “Occluding Device” and U.S. Pat. No. 6,379,329 to Naglreiter entitled, “Detachable Balloon Embolization Device and Method.” Although the presented detachable balloon systems and improvements are numerous, few have been realized as commercial products for aneurysm treatment largely due to an inability to address a majority of the previously mentioned drawbacks.

The most widely used embolization devices are detachable embolization coils. These coils are generally made from biologically inert platinum alloys. To treat an aneurysm, the coils are navigated to the treatment site under fluoroscopic visualization and carefully positioned within the dome of an aneurysm using sophisticated, expensive delivery systems. Typical procedures require the positioning and deployment of multiple embolization coils which are then packed to a sufficient density as to provide a mechanical impediment to flow impingement on the fragile diseased vessel wall. Some of these bare embolization coil systems have been describe in U.S. Pat. No. 5,108,407 to Geremia, et al., entitled, “Method And Apparatus For Placement Of An Embolic Coil” and U.S. Pat. No. 5,122,136 to Guglielmi, et al., entitled, “Endovascular Electrolytically Detachable Guidewire Tip For The Electroformation Of Thrombus In Arteries, Veins, Aneurysms, Vascular Malformations And Arteriovenous Fistulas.” These patents disclose devices for delivering embolic coils at predetermined positions within vessels of the human body in order to treat aneurysms, or alternatively, to occlude the blood vessel at a particular location. Many of these systems, depending on the particular location and geometry of the aneurysm, have been used to treat aneurysms with various levels of success. One drawback associated with the use of bare embolization coils relates to the inability to adequately pack or fill the aneurysm due to the geometry of the coils and their flexibility and conformability which can lead to long term recanalization of the aneurysm with increased risk of rupture.

Some improvements to bare embolization coils have included the incorporation of expandable foams, bioactive materials and hydrogel technology as described in the following U.S. Pat. No. 6,723,108 to Jones, et al., entitled, “Foam Matrix Embolization Device”, U.S. Pat. No. 6,423,085 to Murayama, et al., entitled, “Biodegradable Polymer Coils for Intraluminal Implants” and U.S. Pat. No. 6,238,403 to Greene, et al., entitled, “Filamentous Embolic Device with Expansible Elements.” While some of these improved embolization coils have been moderately successful in preventing or reducing the rupture and re-rupture rate of some aneurysms, the devices have their own drawbacks. For instance, in the case of bioactive coils, the materials eliciting the biological healing response are somewhat difficult to integrate with the coil structure or have mechanical properties incompatible with those of the coil making the devices difficult to accurately position within the aneurysm. In the case of some expandable foam and hydrogel technology, the expansion of the foam or hydrogel is accomplished due to an interaction of the foam or hydrogel with the surrounding blood environment. This expansion may be immediate or time delayed but is generally, at some point, out of the control of the physician. With a time delayed response the physician may find that coils which were initially placed accurately and detached become dislodged during the expansion process leading to subsequent complications.

Other purported improvements to embolization coils such as space filling shapes and the incorporation of polymers, fibers and braid are disclosed in U.S. Pat. No. 5,749,891 to Ken et al., entitled, “Multiple Layered Vaso-occlusive Coils”, U.S. Pat. Nos. 5,226,911 and 5,304,194, both to Chee et al., U.S. Pat. No. 5,382,259, to Phelps et al. and U.S. Pat. No. 6,280,457 to Wallace et al., entitled, “Polymer Covered Vaso-occlusive Devices and Methods of Producing Such Devices.” Ken et al. discloses a device formed from a wire helically wound into a primary coil. A portion of the primary coil is then wound on die forming a large diameter helix creating a first layer. A sheath is placed over the first layer and another portion of the primary coil is wound over the sheath (in the opposite direction) to form a second layer. A second sheath is placed over the second layer and the remaining portion of the primary coil is wound over the sheath (in the opposite direction) to form a third layer. The multiple-layered coil is then heat treated to set the secondary shape of the primary coil. The multiple layered structure of this coil is intended to be more space filling than other single layered prior art coils. The multiple-layered coil may include fibers or braid to increase its occlusive properties. The Phelps et al. patent describes a vaso-occlusive coil which is covered with a polymeric braid on its exterior surface. Wallace et al. discloses various methods and configurations to incorporate polymers into the coils to improve their occlusive properties. One such configuration includes wrapping a small diameter polymer filament directly onto a wire. This polymer wrapped wire is then helically wound to form a primary coil. The primary coil may be shaped into secondary shapes through a heat treatment procedure or the use of a shaped stylet positioned within the lumen of the coil. The wire material properties, diameter of the wire, winding preload (to form the primary coil) and heat treatment to set the shape secondary shape are the major contributing factors to the flexibility and conformability of the multiple-layered coil of Ken et al., the fibered coils of Chee et al., braid covering coils of Phelps et al., and the polymer covered coils of Wallace et al. just like all other prior art coils.

With the aforementioned prior art vaso-occlusion coils a wire is helically wound to form a primary coil that has specific performance characteristics associated directly with the wire diameter and its properties (modulus, hardness, etc.) along with primary coil diameter and its properties (winding pitch, preload, etc.). As one would expect, a primary coil having a certain diameter can be made more flexible by reducing the diameter of the wire used to form the coil (assuming all other variables are held constant). This process of reducing the wire diameter is typically done when making softer and more flexible coils however there is a limit to this process where the formation of a primary coil from very small diameter wire results in a coil that is extremely fragile and unusable for its intended purpose. To extend the usability range of the very small diameter wire, the primary coil diameter is typically reduced to make the primary coil less fragile. However this process also substantially reduces the volume of space that the coils occupy. When occluding a target site, a physician would have to utilize substantially more of these smaller diameter primary coils to occlude the target site, thus increasing the time, cost and risk associated with the procedure. There exists a need for a vaso-occlusion coil having increased flexibility, occupies a large volume and is more durable to reduce costs and risks associated with embolization procedures.

### SUMMARY OF THE INVENTION

In accordance with an aspect of the present invention, there is provided a medical implant that takes the form of an embolization device such as an embolic or vaso-occlusive coil having increased flexibility, durability, conformability and surface area for selective placement within a vessel, aneurysm, duct or other body location. The inventive embolic coils are typically formed through the helical winding of a wire to form an elongate initial coil. The initial coil is then subsequently helically wound to form a primary coil. The primary coil according to an embodiment of the present invention is delivered through a catheter to a target site in a generally linear configuration. The wire or filament is typically a biocompatible material suitable for implantation and includes metals such as platinum, platinum alloys, stainless steel, nitinol and gold. Other biocompatible materials such as plastics groups including nylons, polyesters, polyolefins and fluoro-polymers may be processed to produce suitable filaments for forming initial coils. The wire usually has a circular cross-section, however, non-circular cross-sections, such as “D” shapes, are used in commercially available coils. The diameter of the wire may range from 0.0001″ to about 0.010″ and is largely dependent upon the particular clinical application for the coil. The diameter of the initial coil is generally dependent upon the wire diameter and the diameter of the mandrel used for winding. The initial coil diameter ranges from about 0.001″ to 0.030″ and preferably ranges from 0.0015″ to about 0.015″ and is also dependent upon on the clinical application. The wound initial coil is typically removed from the mandrel leaving the coil with an open lumen. In addition to the aforementioned method of winding an initial coil, there are other “mandrel-less” forming processes that are suitable for making initial coils that plastically deform the wire into an initial coil. The initial coil is then typically wound on a mandrel to form a primary coil. The primary coil is typically removed from the mandrel leaving the primary coil with a lumen. In addition to the aforementioned method of winding a primary coil, there are other “mandrel-less” forming processes that are suitable for making primary coils that plastically deform the initial coil into the primary coil. The formed primary coils may be further processed to have a secondary shape such as a helix, sphere, “flower”, spiral or other complex curved structure suited for implantation in a particular anatomical location. The secondary shape is imparted to the coil through thermal or mechanical means. Thermal means include forming the primary coil into a desired shape using a die or forming tool and then heat treating the primary coil to retain the secondary shape. Mechanical means include plastically deforming the primary coil into the desired shape or the use of a shaped resilient core wire inserted into the lumen of the primary coil to impart a shape to the primary coil. The length of the elongate primary coil range from 0.1 cm to about 150 cm with a preferred range of about 0.5 cm to about 100 cm. The distal end of the primary coil is typically rounded or beaded to make the primary coil end more atraumatic. Other embolic coil modifications suitable for use include the incorporation of a stretch resistant member(s) (within the primary coil lumen or exterior to the coil) that limits undesirable elongation of the primary coil during device manipulation and coated or modified coils that enhance occlusion through coils surface modifications, addition of therapeutics or volume filling materials (foams, hydrogels, etc.).

In accordance with yet another aspect of the present invention there is provided an embolic coil having increased flexibility, conformability and durability that includes a helically wound primary coil formed from a small diameter initial coil which is turn formed from a helically wound biocompatible material and an elongate core element positioned within the lumen of the initial coil. The embolic coil has a structural configuration in which the biocompatible wire or filament characteristics (e.g. diameter, material, etc.) significantly contributes to the flexibility, conformability and durability performance characteristics of the coil. These desirable performance characteristics are typically attained when the ratio of the initial coil diameter to the diameter of the core element is greater than 1.3 and preferably greater than 1.5.

In accordance with another aspect of the present invention there is provided an embolic coil having increased flexibility and conformability and a process of forming the embolic coil from a small diameter initial coil which is helically wound into a primary coil. The initial coil is formed from a small diameter wire which is wound on a sacrificial mandrel or a composite mandrel having a sacrificial portion and a support portion. The wire diameter has a preferable range from about 0.0001″ to about 0.0015″ and more preferable from about 0.0004″ to about 0.00125″. The sacrificial mandrel may be formed of a polymer, metal, ceramic or combinations thereof. The cross sectional shape of the mandrel may be any desirable geometric shape (e.g. round, rectangular, “D”, ribbon, etc.) suitable for winding the initial coil. Once the initial coil is formed on the sacrificial or composite mandrel the initial coil together with the mandrel are wound in a helical fashion about another winding mandrel to form the primary coil. The primary coil winding mandrel may also be of the sacrificial or composite type. The sacrificial or sacrificial portion of the composite mandrel for the initial coil may be removed after forming the primary coil. The primary coil may then be shaped into a secondary shape using thermal or mechanical means. The sacrificial mandrel within the lumen of the initial coil may be removed by thermal decomposition, chemical dissolution or other means. In the case of a composite mandrel having a sacrificial portion and a support portion (e.g., a polymer coated metal wire, a multi filament mandrel having polymer and metal filaments, etc.) the mandrel's polymer components (sacrificial portion) may be removed leaving behind the metal components (support portion) within the lumen of the initial coil. Alternatively, the composite mandrel metal components (sacrificial portion) may be removed leaving behind the polymer components (support portion) within the lumen of the initial coil.

In accordance with yet another aspect of the present invention there is provided an embolic coil having increased flexibility and conformability formed from a small diameter initial coil which is helically wound into a primary coil and includes embolization enhancing materials and configurations. The embolization enhancing materials and configurations may increase the bioactivity (e.g., platelet activation, thrombus formation, cell recruitment, cellular proliferation, etc.) of the embolic coil when compared to bare wire coils. Examples of embolization enhancing materials and configurations include the incorporation of polymer fibers which extend from the coil, braid or mesh coverings over the coil, surface modifications to the coil wire (e.g., plasma deposition, increased surface roughness, etc.) and coatings applied to the coil. Suitable biocompatible coatings include those formed from bio-erodible and or biodegradable synthetic materials. The coating may further comprise one or more pharmaceutical substances or drug compositions for delivering to the tissues adjacent to the site of implantation, and one or more ligands, such as peptides which bind to cell surface receptors, small and/or large molecules, and/or antibodies or combinations thereof for capturing and immobilizing, in particular progenitor endothelial cells on the blood contacting surface of the medical device. Suitable polymer examples of embolization enhancing materials and configurations include polymers such as polyolefins, polyimides, polyamides, fluoropolymers, polyetheretherketone (PEEK), cross-linked PVA hydrogel, polytetrafluoroethylene (PTFE), expanded polytetrafluoroethylene (ePTFE), porous high density polyethylene (HDPE), polyurethane, and polyethylene terephthalate, or biodegradable materials such as polylactide polymers and polyglycolide polymers or copolymers thereof and shape memory polymers. The medical device may comprise numerous materials depending on the intended function of the device.

In accordance with another aspect of the present invention there is provided an embolization system whereby the inventive embolic coil having a primary coil helically wound from an initial coil wound from a helically wound metallic wire is releasably coupled to a delivery system. The embolic coil may be selectively released from the delivery system when delivered to a target site within the body by mechanical, thermo-mechanical, electro-mechanical, hydraulic or electrolytic means.

In accordance with still yet another aspect of the present invention there is provided an embolization system for use in placing an inventive embolic coil at a preselected site within the body of a mammal which includes an elongate delivery system having a coupling assembly at its distal end that releasably engages the proximal end of coil. The delivery system includes an elongate tubular delivery member having proximal and distal ends, a coupling assembly positioned at the distal end of the delivery member and includes an engagement member and a tip member fixedly coupled to the distal end of engagement member. The coupling assembly is releasably coupled to the proximal end of the embolic coil which includes a coupling member having an aperture and an engagement portion. The engagement member of the coupling assembly is positioned within the aperture of the coupling member and the tip member of the engagement member engages the engagement portion of the coupling member. A release member having proximal and distal ends is positioned at the distal end of the delivery member, adjacent to the engagement member. The release member has a first configuration in which the distal end of the release member is positioned within the aperture of the coupling member and in cooperation with the engagement member, restricts the uncoupling of the engagement member from the coupling member. The release member also has a second configuration in which the distal end of the release member is removed from the aperture of the coupling member, thereby allowing the uncoupling of the engagement member from the coupling member.

In accordance with still another aspect of the present invention there is provided an embolic coil deployment system that includes a tubular delivery member having proximal, intermediate and distal regions and comprises multiple zones of flexibility while minimizing the outer diameter profile and reducing the effects of compression and elongation when advancing and retracting the delivery member within a catheter. The tubular delivery member includes a proximal region preferably formed of a multi-filar single layer coil, an intermediate region preferably formed of a multi-filar, multi-layer coil and a distal region formed of a uni-filar coil. The regions of the delivery member may be joined together using known welding techniques including laser and resistance or may be brazed or soldered. The proximal and intermediate regions may alternatively incorporate metallic hypotubes to provide additional strength and minimize system elongation as well as the system profile. The distal region of the delivery member may also include radio opaque marker bands to align with the catheter during delivery and positioning of the embolic coil under fluoroscopy.

In accordance with yet another aspect of the present invention, the release member is positioned within the lumen of the delivery member and the proximal end of the release member extends proximal to the proximal region of the delivery member. The portion of the release member extending proximal to the proximal end of the delivery member may be grasped by a physician and moved proximally relative to the delivery member to move the release member from its first configuration to its second configuration during the release of an implant at the desired site.

In accordance with still yet another aspect of the present invention there is provided a delivery system that includes a proximal spring member positioned proximal to the proximal region of the delivery member. The proximal spring member has proximal and distal ends and is coaxially positioned about the proximal end of the release member such that the release member extends through the lumen of the proximal spring member. The proximal spring member distal end is coupled to the delivery member and the proximal end of the spring member coupled to the proximal end of the release member. The proximal spring member is preferably biased to maintain or place the release member in its first configuration in which the distal end of said release member is positioned within the aperture of the coupling member and in cooperation with the engagement member restrict the uncoupling of the engagement member from the coupling member of the implant. Proximal movement of the spring member proximal end relative to the delivery member causes the release member to move from its first configuration to its second configuration.

### BRIEF DESCRIPTION OF THE DRAWINGS

FIG. 1 is a side view of a medical implant according to an embodiment of the present invention.

FIG. 2 is side view of a medical implant having a complex secondary shape configuration according to another embodiment of the present invention.

FIG. 3 is a side view of the formation of an initial coil according to an embodiment of the present invention.

FIGS. 4A through 4H are cross-sectional views of various geometric shapes and configurations suitable for coil wires and or winding mandrels according to embodiments of the present invention.

FIGS. 5A and 5B are cross-sectional views of configurations for sacrificial mandrels according to yet another embodiment of the present invention.

FIG. 6 is a side view of an initial coil removed from the winding mandrel.

FIG. 7 is a side view of the formation of a primary coil from an initial coil.

FIG. 8 is a side view of the primary coil removed from the winding mandrel.

FIGS. 9A through 9C depict method steps to form coils according to embodiments of the present invention.

FIG. 10A is a partially sectioned side view of an occlusion device with a stretch resistant member and a hollow initial coil.

FIG. 10B is a partially sectioned side view of an occlusion device with a stretch resistant member and an initial coil with a core element disposed within its lumen.

FIG. 11 is a side view of an occlusion device with tufts of fiber positioned along its length.

FIG. 12 is a side view of an occlusion device with a braided covering.

FIG. 13 is a side view of an occlusion device including a bioactive coating.

FIG. 14 is a partially sectioned view of an occlusion device including a proximal coupling.

FIG. 15 is a partially sectioned view of a coil deployment system.

FIGS. 16 through 20 are partial section views illustrating a method of deploying a medical implant within an aneurysm according to an embodiment of the present invention.

### DETAILED DESCRIPTION OF THE INVENTION

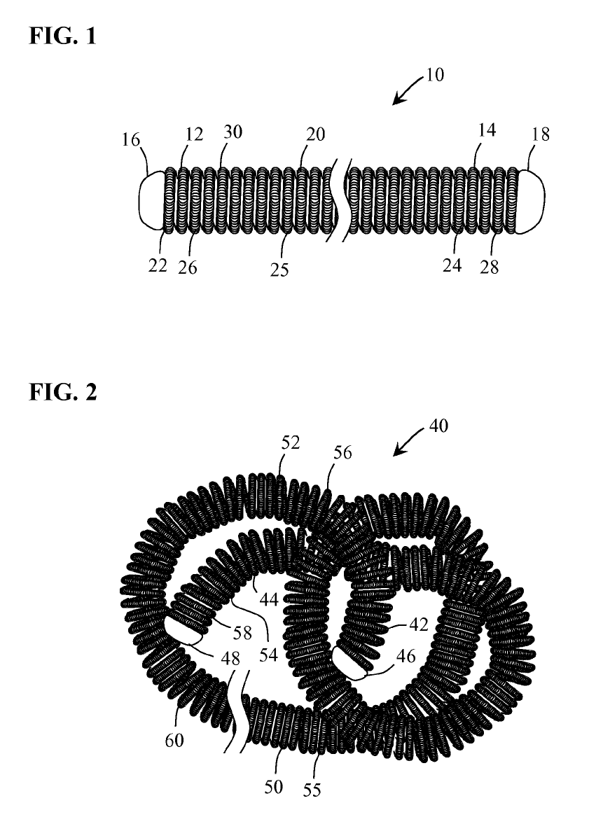

Generally, a medical implant of the present invention may be positioned at a preselected site within lumen of the body of a mammal. More specifically, the medical implant is an occlusion device for use in occluding or restricting fluid flow in ducts, vessels, aneurysms and other areas of the body. FIG. 1 generally illustrates an occlusion device of the present invention that takes the form of an elongate filament-like embolization coil 10 having proximal and distal ends 12, 14 and a central lumen 15. The embolization coil 10 includes atraumatic tips 16, 18 positioned at the proximal and distal ends 12 and 14, respectively. Embolization coil 10 includes a helically wound primary coil 20 having a proximal end 22 and a distal end 24 that is formed from a helically wound initial coil 25 having a proximal end 26 and a distal end 28 which is formed from a biocompatible wire 30.

The atraumatic tips 16 and 18 are shown in a preferred configuration in which they are rounded or beaded. They may be formed by beading the material of the primary coil through the use of a plasma welder, electric arc welder or laser welder. Alternatively the atraumatic tips may be formed through the addition of glue, heat formed polymers or encapsulation with a solder. The atraumatic tips 16 and 18 positioned at the proximal and distal ends of embolization coil 10 preferably have a diameter about equal to the diameter of primary coil 20.

As used herein, when defining dimensional relationships between a first dimension “about equal to” a second dimension the term “about equal to” means that the first dimension may encompass a range of values equal to the second dimension plus or minus 10%. For instance, if the second dimension had a value of 0.015″ then the first dimension “about equal to” the second dimension may have a value within the range of 0.0165″ to 0.0135″.

FIG. 2 depicts another embodiment of an inventive embolization coil 40 which is similar in construction to embolization coil 10. Elongate filamentous embolization coil 40 has proximal and distal ends 42, 44 and a central lumen 45 (not shown). The embolization coil 40 includes atraumatic tips 46, 48 positioned at the proximal and distal ends 42 and 44, respectively. Embolization coil 40 includes a helically wound primary coil 50 having a proximal end 52 and a distal end 54 that is formed from a helically wound initial coil 55 having a proximal end 56 and a distal end 58 which is formed from a biocompatible wire 60. Embolization coil 40 differs from embolization coil 10 in that it has been processed to have a secondary shape when in a relaxed and unconstrained configuration. While embolization coil 40 is shown having a complex curvilinear shape with multiple bends and an overall spheroidal appearance, other geometric shapes including helixes, clovers, cones, boxes, spheres and any combinations thereof are also suitable.

When placed in a generally linear configuration, such as during delivery through a small diameter catheter, the elongate filament-like embolization coils of the present invention have length which is substantially longer than its primary coil diameter, thus a very high length to diameter ratio. This long length enables the coil to occupy a large volume of space when delivered to a target site within the body such as an aneurysm. The construction of the embolization coils allows the inventive coils to have improvements in flexibility and conformability over prior art coils having similar length and primary coil diameters. Prior art coils typically flex only along the helical winds of the primary coil with the wire diameter substantially influencing this ability to bend because the wire must be torqued. Embolization coils of the present invention have the ability to flex along the helical winds of the of the primary coil, however, instead of torqueing a solid wire like the prior art coils, the initial coil can flex in addition to the wire having the torque allowing an additional degree of freedom. This additional ability of the initial coil to flex allows the inventive embolization coils to better conform to wall geometry of a target site and with much lower force than prior art coils. When treating cerebral aneurysms that have a very thin wall, a small diameter or both, the increased flexibility and conformability is especially important in minimizing the potential to rupture the aneurysm during coil placement. Because the inventive embolization coils conform to the irregular geometries often associated with aneurysms better than prior art coils, more space within the aneurysm can be occupied, thereby increasing the packing density of the treated aneurysm leading to more stable occlusions and better long term outcomes.

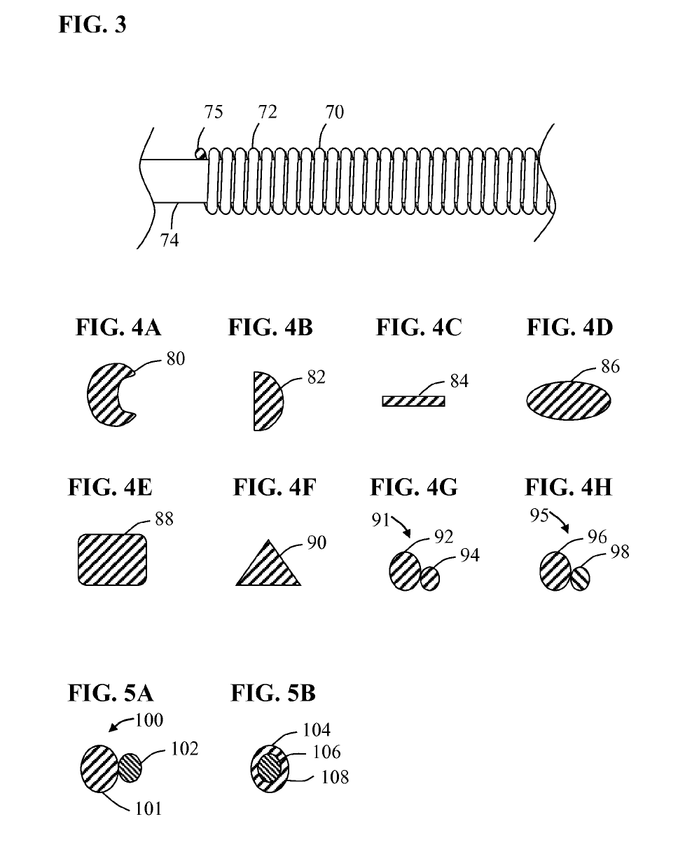

FIGS. 3 through 8 general illustrate base components and accessory tools suitable use in the formation of the inventive embolization coils. FIG. 3 shows an initial coil 70 formed from a biocompatible wire 72 that is helically wound about an elongate cylindrical winding mandrel 74. Biocompatible wire 72 has a cross sectional shape 75 which is shown taking the form of a circle. The biocompatible wire used in forming embolization coils is typically a metallic wire suitable for implantation and includes metals such as platinum, platinum alloys, platinum group metals (e.g. palladium, iridium) and their alloys, tantalum, stainless steel alloys, nitinol and gold. The wire usually has a circular cross-section, however, non-circular cross-sections, such as “D” shapes, may be also suitable. The diameter of the wire may range from 0.0001″ to about 0.010″ and is largely dependent upon the particular clinical application for the embolization coil. While the diameter of the wire is preferably held constant throughout coil length, wire having a varying diameter may also be suitable for varying properties of the embolization coil.

FIGS. 4A through 4H illustrate alternative cross sectional shapes and configurations suitable for biocompatible wires and or winding mandrels, such as biocompatible wire 72 and winding mandrel 74, used to produce coils with distinctive shapes and performance characteristics. FIG. 4A shows a cross sectional shape 80 which is generally in the form of a “C”. FIG. 4B shows a cross sectional shape 82 which is generally in the form of a “D”. FIG. 4C shows a cross sectional shape 84 which is generally in the form of a rectangle. FIG. 4D shows a cross sectional shape 86 which is generally in the form of an ellipse. FIG. 4E shows a cross sectional shape 88 which is generally in the form of a square. FIG. 4F shows a cross sectional shape 90 which is generally in the form of a triangle. FIG. 4G shows a cross sectional configuration 91 that comprises at least two components formed from the same material and includes a cross sectional shape in the form of a large diameter circle 92 adjacent to a cross sectional shape in the form of a small diameter circle 94. FIG. 4H shows a cross sectional configuration 95 that comprises at least two components formed from the different materials and includes cross sectional shape in the form of a large diameter circle 96 adjacent to a cross sectional shape in the form of a small diameter circle 98. As can be appreciated, wires or winding mandrels having cross sectional shapes other round may have a twisted configuration to form coils having a twisted structure along their length. Similarly, wires or winding mandrels having cross sectional configurations including multiple components may be twisted (including those that have a round cross sectional shape) to form coils having a twisted structure along their length.

Typical materials suitable for winding mandrels include metals, ceramics and polymer with preferred materials being stainless steel, nickel titanium alloys and silver plated copper. When winding of any of the coil on a mandrel as indicated, difficulties may be encountered during the removal of the mandrel causing damage to the device. While suitable mandrel materials were previously described, the following processing steps may aid in removing the mandrel from the coil. When using a silver plated copper mandrel, once the winding process is completed, tension may be applied to the ends of mandrel to stretch the mandrel. The process of stretching the mandrel will reduce the cross sectional diameter of the mandrel allowing the coil to more easily slide on the mandrel. Trimming the mandrel in a region that has been reduced in diameter will enable the coil to be removed from the mandrel without damage. When using a preferred mandrel material, such as nitinol, the same process may be used as above to reduce the mandrel cross sectional diameter, however it is preferable to cool the nitinol below its austenitic finish (Af) temperature before stretching to place the mandrel material in a martensitic phase. In the martensitic phase the mandrel is more easily deformable and may be stretched with a lower force to reduce its diameter then when in the austenitic phase. The coil while still on the nitinol mandrel may be placed in suitably cooled fluid (such as an ethanol and dry ice mixture) to cool the assembly below the Af. Once cooled the nitinol mandrel may be stretched, trimmed at a reduced diameter location and quickly removed from the coil. In an alternative process step the nitinol mandrel may be stretched first forming stress induced martensite and while under tension cooled to a temperature below the Af to maintain the mandrel in the martensitic phase for subsequent processing.

Alternatively, mandrels may be used in the formation of the coils which are of the sacrificial type. This type of mandrel may be removed from coil lumen by placing the coil and mandrel in suitable media (e.g. water, acids, bases, organic solvents, etc.) to dissolve the mandrel, and leave behind the intact coil. Dependent upon the particular mandrel material chosen (preferably a polymer), the coil and mandrel may be subjected to heat to thermally decompose or “burn out” the mandrel to also leave behind an intact coil.

FIGS. 5A and 5B illustrate cross sectional configurations for a composite winding mandrel having a sacrificial portion and a non-sacrificial support portion. The cross sectional configuration of composite winding mandrel 100 shown in FIG. 5A, has two components including a sacrificial component 101 and a support component 102. While components 101 and 102 are shown having a circular cross section with differing diameters, they may have the same diameters or any of the cross sectional shapes previously described and also have a twisted configuration. FIG. 5B illustrates a cross sectional configuration of composite winding mandrel 104 having two components including a sacrificial portion 106 and a support portion 108. Support portion 108 is positioned within sacrificial portion 106. In the preferred configuration shown, support portion 108 is concentrically positioned within sacrificial portion 106. While portions 106 and 108 are shown having a circular cross section, they may have the any of the cross sectional shapes previously described. Typical materials suitable for composite winding mandrels include metals, ceramics and polymers with preferred materials being platinum, stainless steel, nickel titanium alloys for the support portions and polyolefins, nylons, polyesters and ultrahigh molecular weight polyethylene for the sacrificial portion. The sacrificial portion of the composite mandrels may be removed by any of the aforementioned techniques discussed for sacrificial mandrels.

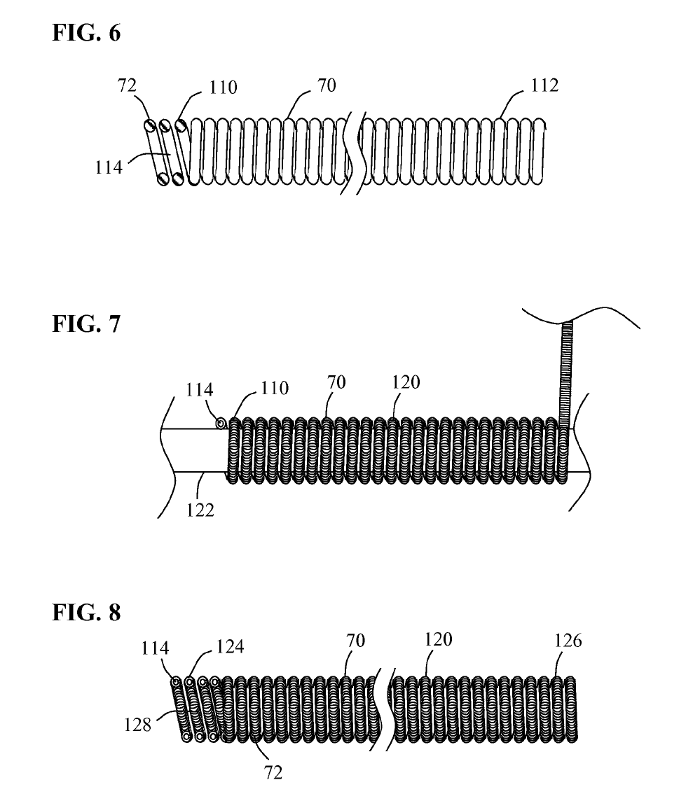

FIG. 6 shows an elongate initial coil 70 formed from a helically wound biocompatible wire 72. Initial coil 70 has a first end 110, a second end 112. The wound initial coil is typically removed from the mandrel leaving the coil with an open lumen 114. In addition to the aforementioned process of winding initial coil 70 on a mandrel, there are other “mandrel-less” forming processes that are suitable for making initial coils that plastically deform the wire into the initial coil. The initial coil diameter typically ranges from about 0.0005″ to 0.030″ and preferably ranges from 0.001″ to about 0.015″ and is dependent upon on the clinical application and geometry of the target site.

Once initial coil 70 has been formed, it can be helically wound to form primary coil 120, as shown in FIG. 7. Typically, initial coil 70 is helically wound about winding mandrel 122. Winding mandrel 122 may be of any type previously discussed (including sacrificial and composite). Winding mandrel 122 may also have a cross sectional shape or configuration according to any of the aforementioned descriptions to produce primary coils that have distinctive shapes and performance characteristics.

FIG. 8 shows an elongate primary coil 120 formed by helically winding initial coil 70 which is in turn formed from a helically wound biocompatible wire 72. Primary coil 120 has a first end 124 and a second end 126. The wound primary coil is typically removed from the mandrel leaving the primary coil with a central lumen 128 extending along the longitudinal axis. Initial coil lumen 114 is in a generally helical configuration about the longitudinal axis and central lumen 128. In addition to the aforementioned process of winding primary coil 120 on a mandrel, there are other “mandrel-less” forming processes that are suitable for making primary coils that plastically deform the initial coil into the primary coil. The primary coil diameter typically ranges from about 0.004″ to 0.250″ and preferably ranges from about 0.006″ to 0.050″ and is dependent upon on the clinical application and geometry of the target site.

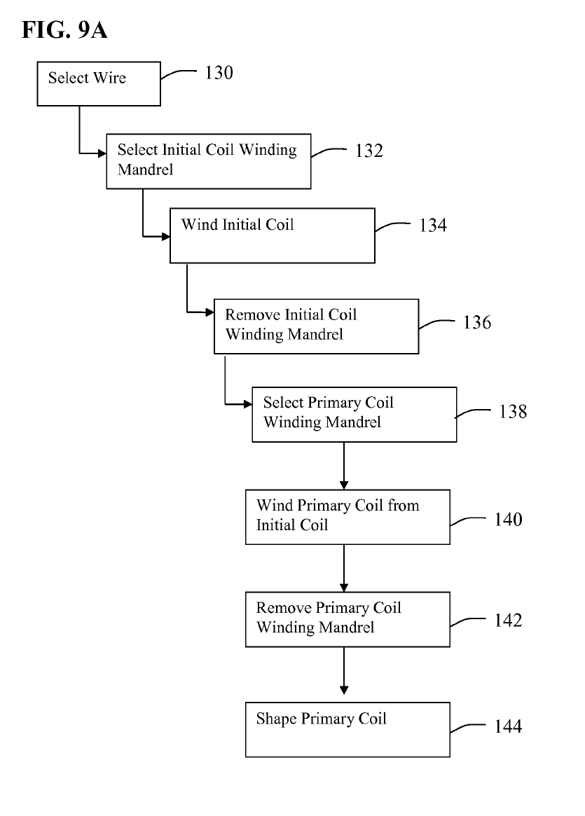

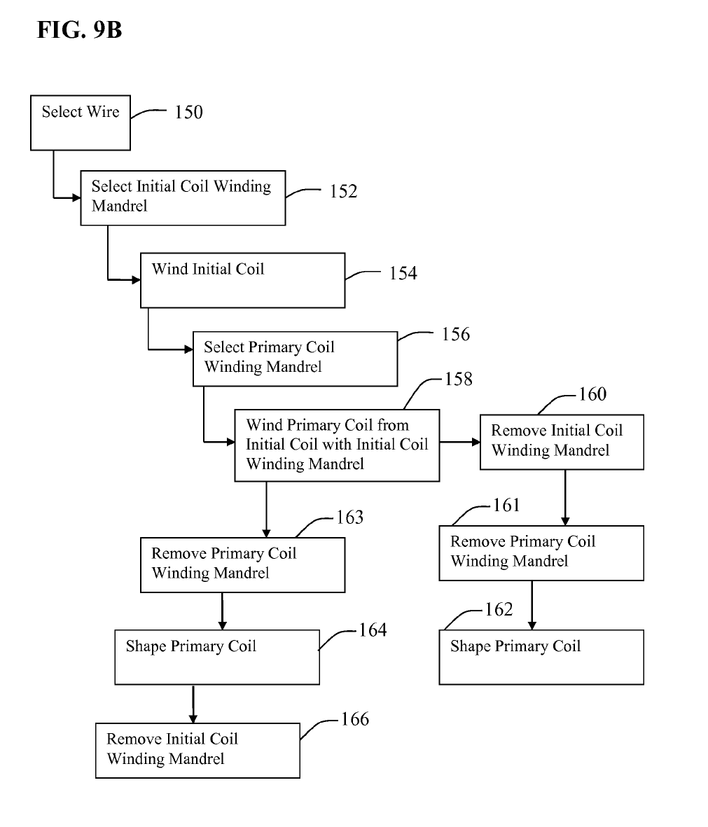

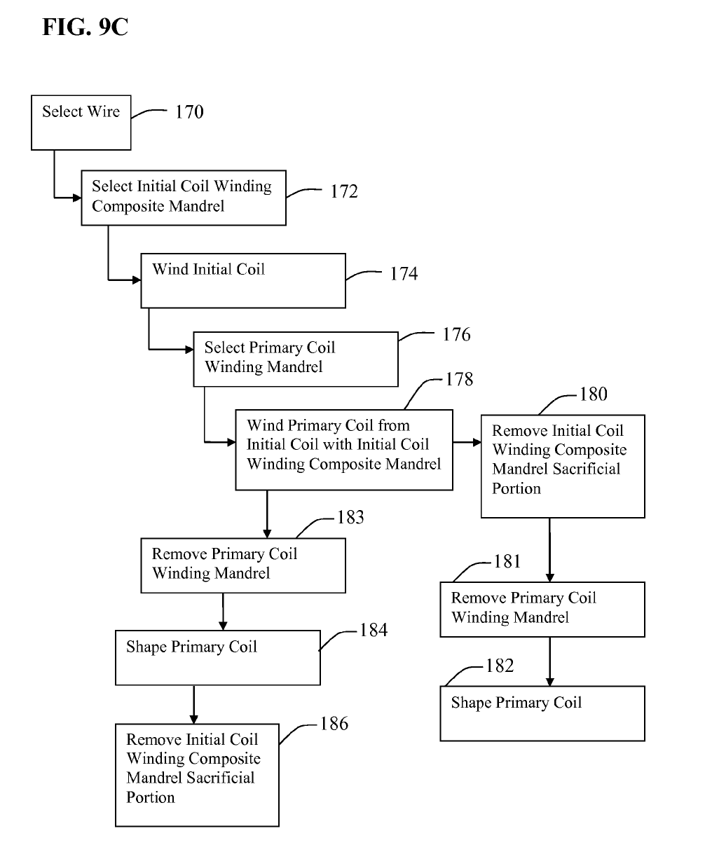

FIGS. 9A through 9C generally list diagrammatic process steps according to embodiments of the present invention to form inventive embolization coils. FIG. 9A shows a general process that includes the step select wire 130. This step generally includes choosing the type of wire and dimensions. The next step in the process is to select initial coil winding mandrel 132. This step includes selecting the initial coil winding mandrel cross sectional shape, configuration, material and dimensions. The next step is to wind initial coil 134. Once the initial coil has been wound, the next step is to remove initial coil mandrel 136. The next steps in the process are select primary coil winding mandrel 138 and wind primary coil from initial coil 140. After the primary coil has been wound the next step is to remove primary coil winding mandrel 142. The primary coil is then ready for secondary operations including the next step which is to shape primary coil 144. Alternatively, when utilizing a mandrel-less coiling process steps 132, 136, 138 and 142 may be omitted.

FIG. 9B shows a general process of forming an emboli coil according to an embodiment of the present invention that includes the step select wire 150. This step generally includes choosing the type of wire and dimensions. The next step in the process is to select initial coil winding mandrel 152. This step includes selecting the initial coil winding mandrel cross sectional shape, configuration, material and dimensions which is a sacrificial winding mandrel. The next step is to wind initial coil 154. Once the initial coil has been wound, the next steps in the process are to select primary coil winding mandrel 156 and wind primary coil from initial coil with initial coil winding mandrel 158. Dependent upon the equipment available, the remove initial coil winding mandrel 160 step may be performed in conjunction with step 158. After the primary coil has been wound the next step is to remove primary coil winding mandrel 161. The primary coil is then ready for secondary operations including the next step which is to shape primary coil 162. Alternatively, after the primary coil has been wound the next step is to remove primary coil winding mandrel 163 while leaving the initial coil winding mandrel within the lumen of the initial coil. The primary coil can then be processed to shape primary coil 164 with subsequently or simultaneously remove initial coil winding mandrel 166.

FIG. 9C shows a general process of forming an emboli coil according to another embodiment of the present invention that includes the step select wire 170. This step generally includes choosing the type of wire and dimensions. The next step in the process is to select initial coil winding composite mandrel 172. This step includes selecting the initial coil winding composite mandrel cross sectional shape, configuration, material and dimensions which has a sacrificial portion and a support portion. The next step is to wind initial coil 174. Once the initial coil has been wound, the next steps in the process are to select primary coil winding mandrel 176 and wind primary coil from initial coil with initial coil winding composite mandrel 178. Dependent upon the equipment available, the remove initial coil winding composite mandrel sacrificial portion 180 step may be performed in conjunction with or subsequent to step 178. After the primary coil has been wound the next step is to remove primary coil winding mandrel 181. The primary coil is then ready for secondary operations including the next step which is to shape primary coil 182. Alternatively, after the primary coil has been wound the next step is to remove primary coil winding mandrel 183 while leaving the initial coil winding composite mandrel within the lumen of the initial coil. The primary coil can then be processed to shape primary coil 164 with subsequently or simultaneously remove initial coil winding composite mandrel sacrificial portion 186 leaving behind the support portion within the lumen of the initial coil. Similarly, the primary coil may be of the composite or sacrificial type and the remove primary coil winding mandrel 183 step performed simultaneously with step 184.

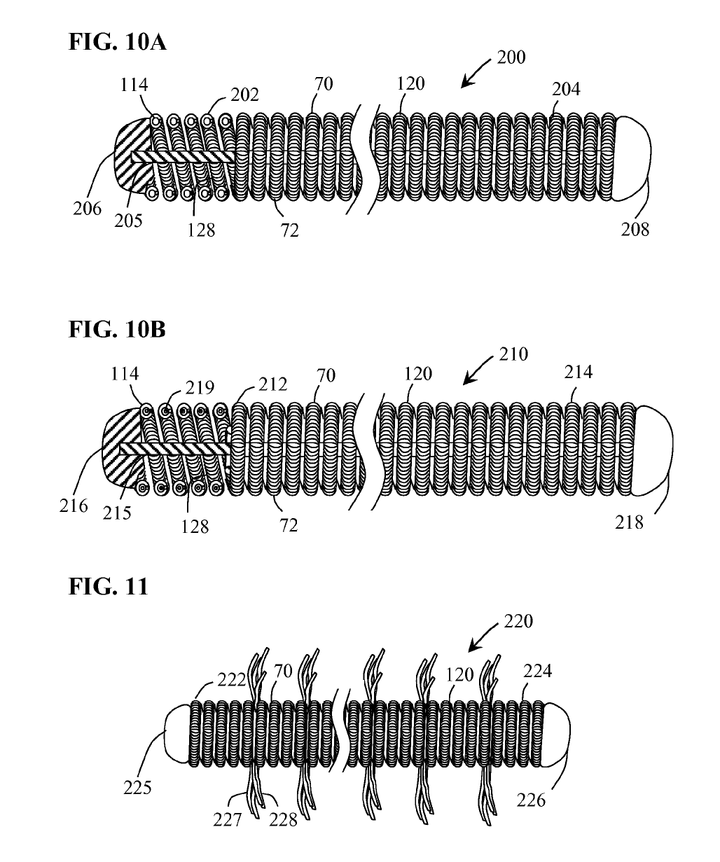

FIG. 10A illustrates an elongate embolic coil 200 according to an embodiment of the present invention. Embolic coil 200 has a proximal end 202 and a distal end 204. Embolic coil 200 is formed from primary coil 120 having a central lumen 128 that extends along the longitudinal axis. As previously described, primary coil 120 is formed by helically winding initial coil 70 which in turn is formed from a helically wound metallic wire 72. Initial coil 70 includes a lumen 114 that extends from proximal end 202 to distal end 204 in a helical fashion about central lumen 128. An elongate stretch resistant member 205 is positioned within lumen 128 of primary coil 120 and extends from proximal end 202 to distal end 204. Stretch resistant member 205 is secured to atraumatic tip 206 located at proximal end 202 and atraumatic tip 208 located at distal end 204. Stretch resistant member is preferably formed of a flexible material and is configured to limit the undesirable stretching of the embolic coil during use. Apart from the influence of stretch resistant member 205, the performance characteristics (flexibility, durability and conformability) of embolic coil 200 are largely dependent upon wire 72, initial coil 70 and primary coil 120 characteristics that include the wire material, wire dimensions, initial coil dimensions and primary coil dimensions.

FIG. 10B illustrates another elongate embolic coil 210, similar in construction to elongate embolic coil 200, according to an embodiment of the present invention. Embolic coil 210 has a proximal end 212 and a distal end 214. Embolic coil 210 is formed from primary coil 120 having a central lumen 128 that extends along the longitudinal axis. As previously described, primary coil 120 is formed by helically winding initial coil 70 which in turn is formed from a helically wound metallic wire 72. Initial coil 70 includes a hollow lumen 114 that extends from proximal end 212 to distal end 214 in a helical fashion about central lumen 128. An elongate stretch resistant member 215 is positioned within lumen 128 of primary coil 120 and extends from proximal end 212 to distal end 214. Stretch resistant member 215 is secured to atraumatic tip 216 located at proximal end 212 and atraumatic tip 218 located at distal end 214. Stretch resistant member is preferably formed of a flexible material and limits undesirable stretching of the embolic coil during use. An elongate support member 219 is positioned within lumen 114 of initial coil 70 and typically extends from proximal end 212 to distal end 214 of embolic coil 210. Support member 219 aids in the durability of the coil by keeping the initial coil from being crushed when the initial coil is formed from very small diameter wire. The support member is preferably formed from a resilient material and generally includes metals or polymers with nitinol being preferred. Apart from the influence of stretch resistant member 215, the performance characteristics (flexibility, durability and conformability) of embolic coil 210 are largely dependent upon support member 219, wire 72, initial coil 70 and primary coil 120 characteristics that include the support member material, support member dimensions, wire material, wire dimensions, initial coil dimensions and primary coil dimensions.

In a preferred embodiment of the embolic coil, the embolic coil mechanical performance includes a mixture of the mechanical performance contributions from the support member and the initial coil forming wire where both components make significant contributions (greater than about 15%) to the overall mechanical performance. Typically, the support member diameter to the wire diameter should have a ratio that ranges from about 5 to about 0.8 and preferably from about 4 to about 1. This range may be increased to about 7 to 1, in special instances, for example when the support member is formed of a polymer and the coil wire is a metal. When the ratio is outside of this range the embolic coil mechanical performance of the embolic coil is substantially determined by either the support member or the initial coil forming wire.

In another preferred embodiment, the embolic coil mechanical performance includes a mixture of the mechanical performance contributions from the support member and the initial coil where both components make significant contributions (greater than about 15%) to the embolic coils mechanical performance. Typically, the initial coil diameter to support member diameter ratio is greater than 1.3, preferably greater than 1.5 and most preferably greater than 2. This ratio provides balanced performance characteristics for flexibility, and durability. When the initial coil diameter to support member diameter ratio is greater than about 10, the durability of the embolic coil can become reduced when very small wire diameters (less than about 0.00125″) are used to form the initial coil.

To improve the occlusion performance of the inventive embolic coils, polymer fibers may be incorporated in the coil. FIG. 11 depicts an embolic coil 220 similar in construction to elongate embolic coils 200 and 210, according to another embodiment of the present invention. Embolic coil 220 has a proximal end 222 and a distal end 224. Embolic coil 220 is formed from primary coil 120 having a central lumen 128 that extends along the longitudinal axis. As previously described, primary coil 120 is formed by helically winding initial coil 70 which in turn is formed from a helically wound metallic wire 72. Embolic coil 220 may include a stretch resistant member that extends from the proximal end to the distal end and or a support member positioned within the lumen of the initial coil as with some the aforementioned embolic coils. Embolic coil 220 includes atraumatic tip 225 located at proximal end 222 and atraumatic tip 226 located at distal end 224 to minimize injury to tissue during implantation. The occlusion properties of embolic coil 220 are enhanced by positioning a plurality of fiber tufts 227 along the coil length or portion thereof. Each fiber tuft 227 preferably contains multiple polymeric fibers 228, arrange such that they extend outwardly from the outer diameter of embolic coil 220. There are numerous ways in which the fibers may be coupled to the inventive coil, such as being compressively held between adjacent turns or winds of primary coil 120. Fibers 228 typically have a very small diameter and typically fold when delivered through the lumen of a small diameter catheter. The fibers are typically made of any biocompatible material such as metals ceramics/glasses and polymers, however polymers are preferred. Suitable polymer examples include polymers such as polyolefins, polyimides, polyamides, fluoropolymers, polyetheretherketone (PEEK), hydrogels cross-linked PVA hydrogel, polytetrafluoroethylene (PTFE), expanded polytetrafluoroethylene (ePTFE), porous high density polyethylene (HDPE), polyurethane, and polyethylene terephthalate, or biodegradable materials such as polylactide polymers and polyglycolide polymers or copolymers thereof and shape memory polymers.

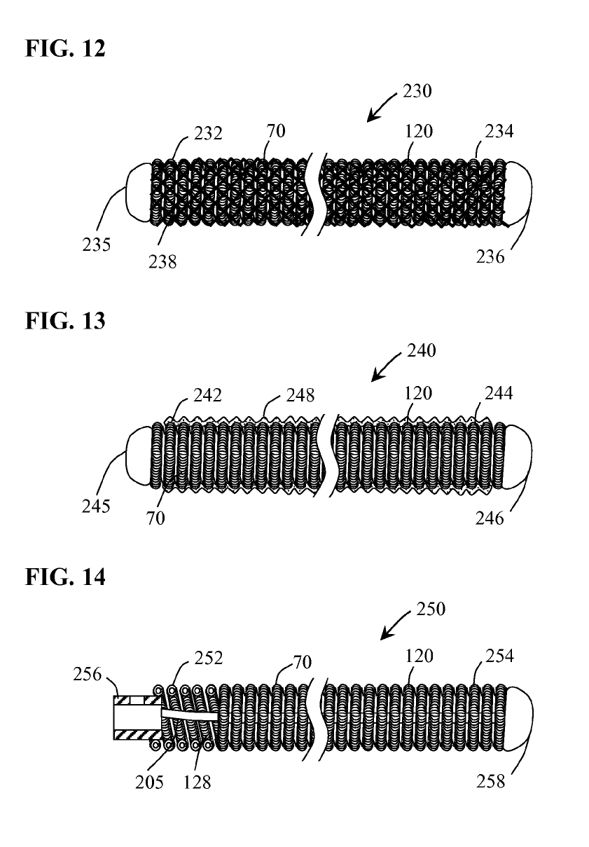

FIG. 12 shows inventive embolic coil 230 according to another embodiment of the present invention which is similar in construction to elongate embolic coils 200 and 210. Embolic coil 230 has a proximal end 232 and a distal end 234. Embolic coil 230 is formed from primary coil 130 having a central lumen 138 that extends along the longitudinal axis. As previously described, primary coil 120 is formed by helically winding initial coil 70 which in turn is formed from a helically wound metallic wire 72. Embolic coil 230 includes atraumatic tip 235 located at proximal end 232 and atraumatic tip 236 located at distal end 234 to minimize injury to tissue during implantation. Embolic coil 230 may include a stretch resistant member that extends from the proximal end to the distal end and or a support member positioned within the lumen of the initial coil as with some the aforementioned embolic coils. To improve the occlusive properties of embolic coil 230, a mesh like covering 238 is positioned on the exterior of primary coil 120. Mesh like covering 238 may take the form of braided fibers, laser cut tubes, perforated metallic thin film sheeting and the like. Mesh like covering 238 may be formed of resilient materials providing an expanded configuration larger than the primary coil outer diameter (not shown). Materials suitable for mesh like covering 238 include biocompatible metals and polymers, such as gold, nitinol, polyolefins, polyimides, polyamides, fluoropolymers, polyetheretherketone (PEEK), hydrogels cross-linked PVA hydrogel, polytetrafluoroethylene (PTFE), expanded polytetrafluoroethylene (ePTFE), porous high density polyethylene (HDPE), polyurethane, and polyethylene terephthalate, polylactide polymers and polyglycolide polymers or combinations thereof.

Biocompatible coatings may be applied to the inventive embolic coils to improve the occlusive properties or healing response associated with the implantation the coils as shown in FIG. 13. Embolic coil 240, similar in construction to embolic coils 200 and 210, include coating 248 positioned on the exterior of primary coil 120. The coating may extend to the interior of the coil if so desired (not shown). Coating 248 may take different forms and include biocompatible coatings which are non-erodible or non-degradable, bio-erodible or biodegradable or combinations thereof. The coating may further comprise or incorporate one or more pharmaceutical substances or drug compositions for delivery to the tissues adjacent to the site of implantation, and one or more ligands, such as peptides which bind to cell surface receptors, small and/or large molecules, and/or antibodies or combinations thereof for capturing and immobilizing, in particular progenitor endothelial cells on the blood contacting surface of the medical device.

Another embodiment of the present invention is shown in FIG. 14, which takes the form of embolic coil 250, which is also similar to construction to embolic coils 200 and 210. Embolic coil 250 is formed from primary coil 120 having a central lumen 128 that extends along the longitudinal axis. As previously described, primary coil 120 is formed by helically winding initial coil 70 which in turn is formed from a helically wound metallic wire 72. Initial coil 70 includes a lumen 114 that extends from proximal end 252 to distal end 254 in a helical fashion about central lumen 128. An elongate stretch resistant member 205 is positioned within lumen 128 of primary coil 120 and extends from proximal end 202 to distal end 204. Stretch resistant member 205 is secured to coupling member 256 located at proximal end 252 and atraumatic tip 258 located at distal end 254. Coupling member 256 is adapted to be releasably coupled to a delivery system. Coupling member 256 may take many different forms dependent upon the mode of operation of the release system. Examples of release systems that can uncouple from the coupling member include those which may be activated through mechanical means, thermo-mechanical, hydraulic mechanical means, electro-mechanical means, electro-chemical means, chemical means and electrolytic means.

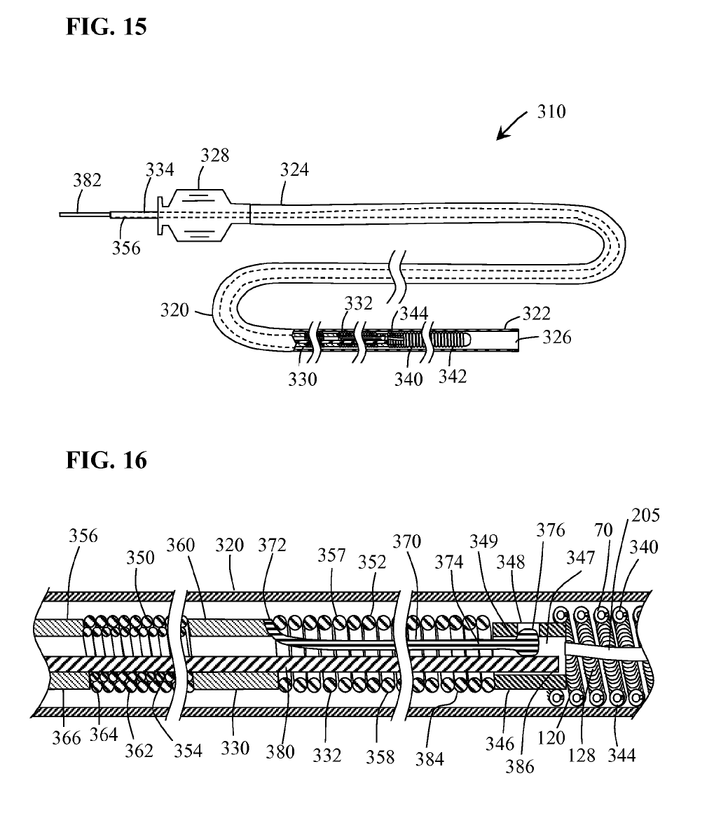

FIG. 15 generally illustrates an embolic coil deployment system 310 according to another embodiment of the present invention which includes delivery catheter 320 having a distal end 322, a proximal end 324, a lumen 326 extending therethrough and a catheter hub 328 affixed to proximal end 324, a delivery system 330 having a distal end 332 and a proximal end 334 and an embolic coil 340 having a distal end 342 and a proximal end 344 that is releasably coupled to the distal end 332 of delivery system 330. Embolic coil 340 is a medical implant of a general type suitable for use in occluding a vessel, lumen, duct or aneurysm.

Embolic coil 340 is similar in construction to embolic coils 200, 210 and 250 and formed from primary coil 120 having a central lumen 128 that extends along the longitudinal axis. As previously described, primary coil 120 is formed by helically winding initial coil 70 which in turn is formed from a helically wound metallic wire 72. Initial coil 70 includes a lumen 114 that extends from proximal end 344 to distal end 342 in a helical fashion about central lumen 128. An elongate stretch resistant member 205 is positioned within lumen 128 of primary coil 120 and extends from proximal end 344 to distal end 342. Stretch resistant member 205 is secured to atraumatic tip 345 located at distal end 342 and coupling member 346 located at proximal end 344. Helically wound wire 72 is made from a material which is biocompatible and preferably radio-opaque. Suitable biocompatible materials include metals such as platinum, platinum alloys, platinum group metals (e.g. palladium, iridium) and their alloys, tantalum, stainless steel alloys, nitinol and gold. As previously discussed, the formed primary coils may be further processed to have a secondary shape such as a helix, sphere, “flower”, spiral or other complex curved structure suited for implantation in a particular anatomical location. The secondary shape is imparted to the coil through thermal and or mechanical means. Thermal means include forming the primary coil into a desired shape using a die or forming tool and then heat treating the coil to retain the secondary shape. Mechanical means include plastically deforming the primary coil into the desired shape or the use of a shaped resilient core wire inserted into the lumen of the primary coil to impart a shape to the coil. The length of the elongate primary coil ranges from 0.1 cm to about 150 cm with a preferred range of about 0.5 cm to about 100 cm. The distal end of the coil is typically rounded or beaded to make the coil end more atraumatic. Other variations of embolic coils suitable for use include stretch resistant coils, coils that incorporate a stretch resistant member(s) (within the central coil lumen or exterior to the coil) that limit undesirable elongation of the primary coil during device manipulation and coated or modified coils that enhance occlusion through coil surface modifications, addition of therapeutics or volume filling materials (foams, hydrogels, etc.).

FIG. 16 illustrates in more detail the construction of the embolic coil deployment system 310 with the implant, coil 340, being positioned within lumen 326 of catheter 320. Embolic coil 340 includes a generally tubular headpiece coupling member 346 positioned at coil proximal end 344. Headpiece coupling member 346 includes a first aperture 347 extending longitudinally, a second aperture 348 extending through the tubular wall and an engagement portion 349. Delivery system 330 includes a tubular delivery member 350 having a distal region 352, an intermediate region 354, a proximal region 356 and a lumen 357 extending therethrough. Distal region 352 of delivery member 350 preferably takes the form of a helically wound coil 358 having a wire diameter ranging from 0.0005 in to 0.006 in and a preferred wire diameter range of about 0.001 in to 0.003 in. Distal region 352 has an axial length that ranges from about 1 cm to about 10 cm and preferably ranges from about 2.5 cm to about 3.5 cm. Tubular marker band 360 is coupled to the proximal portion of coil 358. Intermediate region 354 of delivery member 350 preferably takes the form of a multi-filar wound coil having an outer coil 362 having a number of filaments ranging from 5 to 12 and with filament diameters ranging from 0.001 in to 0.005 in and a preferred number of filaments ranging from 6 to 8 and preferred filament diameters between about 0.0015 in and 0.0035 in and an inner coil 364 having a number of filaments ranging from 5 to 12 and with filament diameters ranging from 0.001 in to 0.005 in and a preferred number of filaments ranging from 6 to 8 and preferred filament diameters between about 0.0015 in and 0.0035 in. Intermediate region 354 has an axial length that ranges from about 20 cm to about 50 cm and preferably ranges from about 30 cm to about 40 cm. The distal ends of intermediate region coils 362 and 364 are preferably welded to the proximal end of marker band 360. Proximal region 356 of delivery member 350 preferably takes the form of a hypotube having a wall 366. Proximal region 356 has an axial length that ranges from about 120 cm to about 170 cm and preferably ranges from about 140 cm to about 160 cm. The distal end of wall 366 is preferably welded to the proximal end of intermediate region 354. While the aforementioned distal, intermediate, proximal regions of delivery member 350 are presented with their respective preferred forms to produce a delivery member having a small diameter profile, these regions of delivery member 350 may also take the form of components used in the construction of catheters and microcatheters, that include laser cut hypotubes, standard hypotubes, braided materials, tubular polymer materials and composites.

Delivery system 330 also includes an engagement member 370 having a proximal end 372, a distal end 374 and a tip member 376 coupled to distal end 374. Tip member 376 preferably takes the form of a generally spherical bead, however, shapes such as rounded disks and other curvilinear geometries that allow the tip member to easily disengage from the engagement portion of the implant coupling member may also be suitable. Engagement member 370 is shown positioned at the distal region 352 of delivery member 350 and secured to delivery member 350 preferably by laser welding but may take the form of any suitable joining technique such as soldering, spot welding, adhesives and ultrasonic welding. Delivery system 330 also includes an elongate release member 380 having a proximal end 382, a distal end 384 and a tip portion 386. Release member 380 preferably takes the form of an elongate resilient nitinol wire which has a lubricious coating although other materials such as stainless steel, platinum alloys, glass or ceramic fibers, polymeric fibers, etc. and forms such as tubes or cables may be suitable. Release member 380 typically has a length which is longer than the combined lengths of the distal, intermediate and proximal regions of delivery system 330. Release member 380 is positioned within lumen 357 of delivery member 350 where the proximal end 382 extends proximal to proximal region 356.

As previously discussed, the proximal end 344 of embolic coil 340 is releasably coupled to the distal end 332 of delivery system 330. More particularly, delivery member distal region 352 and engagement member 370 engage coupling member 346 positioned at coil proximal end 344. As shown in FIG. 16, the distal end 374 of engagement member 370 is positioned within aperture 347 of coupling member 346. Aperture 347 has a diameter larger than the diameter of tip member 376, thereby allowing tip member 376 to be easily inserted into or removed from coupling member 346. In a first configuration, distal end 384 of release member 380 is positioned within aperture 347 of coupling member 346 adjacent to engagement member distal end 374, while tip member 376, is partially positioned within aperture 348 and is engaged with engagement portion 349. The diameters of release member distal end 384 and engagement member distal end 374 cooperatively restrict tip member 376 from being withdrawn through aperture 347. While in this first configuration, coil proximal end 344 is securely coupled to delivery member distal region 352 and allows delivery member 350 to advance or retract embolic coil 340 within the catheter. In a second configuration, distal end 384 of release member 380 is withdrawn from aperture 347 of coupling member 346 allowing tip member 376 of engagement member distal end 374 to be removed from aperture 348 and disengage from engagement portion 349. The removal of release member distal end 384 from aperture 347 allows tip member tip member 376 to be withdrawn from aperture 347 thereby uncoupling coil 340 from the delivery member.

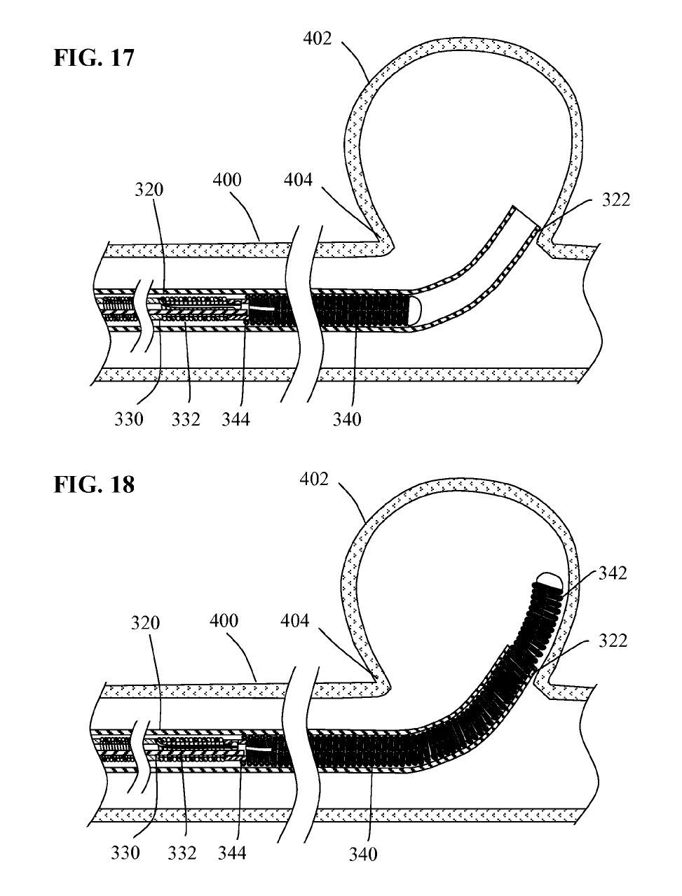

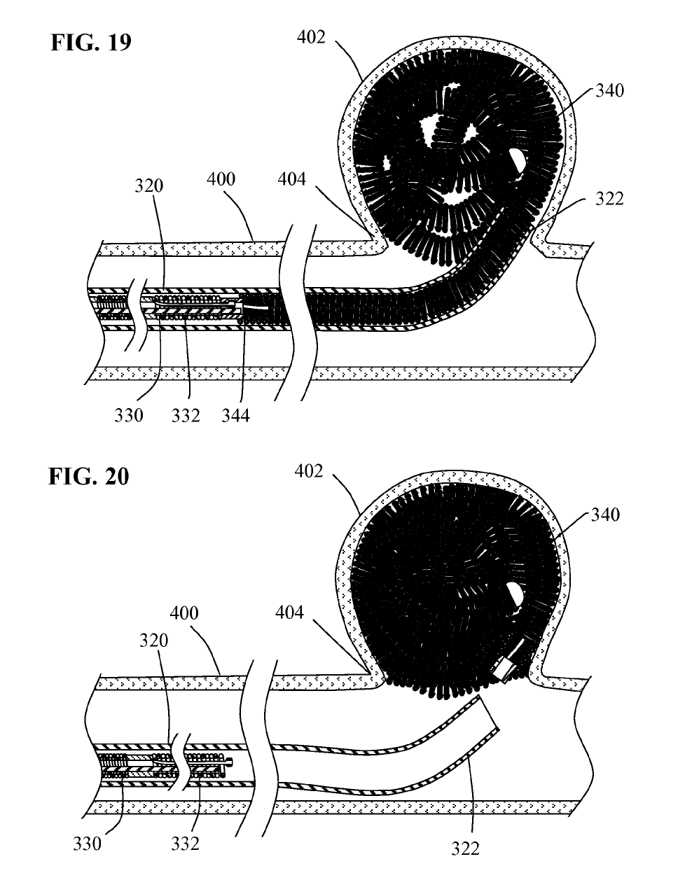

FIGS. 17 through 20 illustrate the method steps of using embolic coil deployment system 310 to treat an aneurysm of a blood vessel. Embolic coil deployment system 310 is inserted into blood vessel 400 and catheter 320 is moved to a position within vessel 400 where catheter distal end 322 is positioned within aneurysm 402 adjacent to aneurysm neck 404 (FIG. 17). Embolic coil 340 is inserted into the lumen of catheter 320 and has a generally linear configuration. Delivery system 330, coupled to embolic coil 340 with release member 380 in a first configuration, is advanced distally within catheter 320 such that embolic coil 340 begins to exit catheter lumen 326 and enter aneurysm 402 (FIG. 18). Further advancement of delivery system 330 allows embolic coil 340, which is capable of folding upon its self, to take a shape within aneurysm 402 with embolic coil 340 forming a scaffold or framework (FIG. 19). Because of the improved flexibility and conformability of embolic coil 340, the aneurysm may be filled to a higher packing density than with prior art coils. During delivery, the physician may retract and advance delivery system 330 to reposition embolic coil 340 into the desired scaffold geometry. Due to the increased durability of the inventive coils, the physician may reposition these coils multiple times (if needed) while reducing damage imparted to the coils as compared to prior art coils. Once embolic coil 340 is properly positioned within aneurysm 402, release member 380 is moved to its second configuration, thereby uncoupling delivery system 330 from embolic coil 340. Delivery system 330 may then be removed from catheter 320 and the body. If the volume filling of the aneurysm is determined to be insufficient, the physician may deploy another embolic coil into the aneurysm and fill to achieve the desired packing density, otherwise catheter 320 can be removed (FIG. 20). With the inventive embolic coil 340 positioned within the aneurysm and across the aneurysm neck, the increased surface area and structure, due to the winds of the initial coil, provide an excellent scaffold for cell proliferation and tissue organization leading to a stable long term occlusion.

As is apparent, there are numerous modifications of the embodiments described above which will become readily apparent to one skilled in the art. It should be understood that various modifications including the substitution of elements or components which perform substantially the same function in the same way to achieve substantially the same result may be made by those skilled in the art without departing from the scope of the claims which follow.

1.-50. (canceled)

51. A medical implant system for occluding at least a portion of a body lumen including:
an elongate flexible catheter having a proximal end, a distal end and lumen extending therethrough;
an embolization device having proximal and distal ends comprising an elongate primary coil having proximal and distal ends and a central lumen extending between said proximal and distal ends, said primary coil being constructed from a plurality of helically wound turns of an initial coil having proximal and distal ends and a lumen extending between said proximal and distal ends, said initial coil being constructed from a plurality of helically wound turns of a biocompatible metallic wire, said primary coil having a delivery configuration positioned within the lumen of said catheter wherein said primary coil is substantially linear and said initial coil is substantially helical; and,
an elongate delivery system slidably positioned within the lumen of said catheter including a pusher member having proximal and distal ends wherein the distal end of said pusher member is releasably coupled to said embolization device, said delivery system having a first configuration coupled to said embolization device and a second configuration wherein said delivery system is uncoupled to said embolization device, said delivery system being selectively operable between said first and second configuration such that when in said first configuration and said embolization device is positioned at a desired location, said delivery system may be operated to place the delivery system in the second configuration thereby releasing the embolization device at the desired location.

52. An embolization device according to claim 51 further including a stretch resistant member positioned within said central lumen.

53. An embolization device according to claim 51 wherein said elongate primary coil includes a core element positioned within said central lumen and said core element imparts a secondary shape to said primary coil.

54. An embolization device according to claim 51 wherein said elongate primary coil includes a braided covering over an outer surface of said primary coil.

55. An embolization device according to claim 51 wherein the lumen of said initial coil is hollow.

56. An embolization device according to claim 51 wherein said initial coil includes a support member positioned within the lumen of said initial coil.

57. An embolization device according to claim 51 wherein said initial coil includes a support member having an outer surface is positioned within the lumen of said initial coil and the metallic wire of said initial coil at least partially resides within indentations on the outer surface of said support member.

58. An embolization device according to claim 51 wherein said primary coil includes a bioactive coating.

59. An embolization device according to claim 51 wherein said primary coil includes a plurality of fibers which extend outwardly from an outer surface of said primary coil.

60. An embolization device according to claim 51 wherein said primary coil has a secondary shape.

61. An intralumenal occlusion device comprising:
an elongate primary coil having a longitudinal axis, proximal and distal ends and a central lumen extending between said proximal and distal ends, said primary coil being constructed from a plurality of helically wound turns of an initial coil having proximal and distal ends, a diameter and a lumen extending between said proximal and distal ends, said initial coil being constructed from a plurality of helically wound turns of a biocompatible metallic wire having a diameter, said primary coil having a delivery configuration wherein said primary coil is generally linear and said initial coil is substantially helical when positioned within the lumen of a catheter; and
an elongate core element formed from a resilient material having a proximal end, a distal end and a diameter, said core element being positioned within the lumen of said initial coil and the diameter of said core element to the diameter of said metallic wire has a ratio between 7 and 0.8.

62. An intralumenal occlusion device according to claim 61 wherein said elongate primary coil includes a stretch resistant member extending through said central lumen.

63. An intralumenal occlusion device according to claim 61 wherein said elongate primary coil includes a braided covering over an outer surface of said primary coil.

64. An intralumenal occlusion device according to claim 61 wherein said primary coil includes a bioactive coating.

65. An intralumenal occlusion device according to claim 61 wherein said primary coil has a secondary shape.

66. An intraluminal occlusion device according to claim 61 wherein said ratio is between 5 and 0.8.

67. An intralumenal occlusion device comprising:
an elongate primary coil having a longitudinal axis, proximal and distal ends and a central lumen extending between said proximal and distal ends, said primary coil being constructed from a plurality of helically wound turns of an initial coil having proximal and distal ends, a diameter and a lumen extending between said proximal and distal ends, said initial coil being constructed from a plurality of helically wound turns of a biocompatible metallic wire, said primary coil having a delivery configuration wherein said primary coil is generally linear and said initial coil is substantially helical when positioned within the lumen of a catheter; and
an elongate core element formed from a resilient material having a proximal end, a distal end and a diameter, said core element being positioned within the lumen of said initial coil and the diameter of said initial coil to the diameter of said core element has a ratio greater than 1.5.

68. An intralumenal occlusion device according to claim 67 wherein said elongate primary coil includes a braided covering over an outer surface of said primary coil.

69. An intralumenal occlusion device according to claim 67 wherein said primary coil includes a bioactive coating.

70. An intraluminal occlusion device according to claim 67 wherein said ratio is greater than 2.
